(19) United States
(12) Patent Application Publication
(10) Pub. No.: US 2005/0239754 A1
(43) Pub. Date: Oct. 27, 2005

(54) S-[2-[(1-Iminoethyl)amino]ethyl]-2-methyl-L-cysteine salicylate monohydrate crystalline salt

(76) Inventor: Lyle Brostrom, Lincolnshire, IL (US)

Correspondence Address: Pharmacia Corporation;Corporate Patent Department, P.O. Box 1027, Chesterfield, MO, 63006, US

(21) Appl. No.: 10/797,349

(22) Filed: Mar. 10, 2004

Related U.S. Application Data
(60) Provisional application No. 60/453,772, filed on Mar. 11, 2003.

(51) Int. Cl. A61K 31/60

(52) U.S. Cl. 514/159; 562/477

(57) ABSTRACT

A crystalline salicylate monohydrate salt of S-[2-[(1-Iminoethyl)amino]ethyl]-2-methyl-L-cysteine is disclosed. A method to make crystalline S-[2-[(1-Iminoethyl)amino]ethyl]-2-methyl-L-cysteine is further disclosed. In addition, methods of use for crystalline S-[2-[(1-Iminoethyl)amino]ethyl]-2-methyl-L-cysteine are disclosed

### FIELD OF THE INVENTION

Priority is claimed from U.S. Provisional Application Ser. No. 60/453,772, filed Mar. 11, 2003 incorporated herein by reference

The present invention comprises a novel compound useful in the treatment of disease, and more particularly a novel salt of S-[2-[(1-Iminoethyl)amino]ethyl]-2-methyl-L-cysteine, and pharmaceutical compositions thereof, for the treatment of conditions involving an inappropriate expression of nitric oxide from the inducible isoform of nitric oxide synthase.

S-[2-[(1-Iminoethyl)amino]ethyl]-2-methyl-L-cysteine is described and claimed in commonly assigned U.S. Pat. No. 6,403,830, herein incorporated by reference.

### BACKGROUND OF THE INVENTION

Nitric oxide (NO) is a bioactive free radical gas produced by any one of several isoforms of the enzyme nitric oxide synthase (NOS). The physiological activity of what was later identified as NO was initially discovered in the early 1980's when it was found that vascular relaxation caused by acetylcholine is dependent on the presence of the vascular endothelium. The factor derived from the endothelium, then called endothelium-derived relaxing factor (EDRF), that mediates such vascular relaxation is now known to be NO that is generated in the vascular endothelium by one isoform of NOS. The activity of NO as a vasodilator has been known for well over 100 years. In addition, NO is the active species derived from known nitrovasodilators including amylnitrite, and glyceryltrinitrate. Nitric oxide is also an endogenous stimulator of soluble guanylate cyclase (cGMP), and thus stimulates cGMP production. When NOS is inhibited by N-monomethylarginine (L-NMMA), cGMP formation is completely prevented. In addition to endothelium-dependent relaxation, NO is known to be involved in a number of biological actions including cytotoxicity of phagocytic cells and cell-to-cell communication in the central nervous system.

The identification of EDRF as NO coincided with the discovery of a biochemical pathway by which NO is synthesized from the amino acid L-arginine by the enzyme NO synthase. There are at least three types of NO synthase as follows:
(i) a constitutive, Ca++/calmodulin dependent enzyme, located in the brain, that releases NO in response to receptor or physical stimulation;
(ii) a Ca++ independent enzyme, a 130 kD protein, which is induced after activation of vascular smooth muscle, macrophages, endothelial cells, and a number of other cells by endotoxin and cytokines; and
(iii) a constitutive, Ca++/calmodulin dependent enzyme, located in the endothelium, that releases NO in response to receptor or physical stimulation.

Once expressed, inducible nitric oxide synthase (hereinafter “iNOS”) generates NO continuously for long periods. Clinical studies have shown that NO production and iNOS expression are increased in a variety of chronic inflammatory diseases, such as rheumatoid and osteoarthritis (see, e.g, McInnes I. B. et al., J. Exp. Med. 184:1519 (1996)), inflammatory bowel disease (see, e.g, Lundberg J. O. N. et al., Lancet 344:1673, (1994)), and asthma (see, e.g., Hamid, Q. et al., Lancet 342:1510 (1993)), and iNOS is implicated as a major pathological factor in these chronic inflammatory diseases.

Thus, inhibition of excessive NO production by iNOS is likely to be anti-inflammatory. However, since the production of NO from eNOS and nNOS is involved in normal physiology, it would be desirable for any NOS inhibitor that is used for treating inflammation be selective for iNOS, so that normal physiological modulation of blood pressure by eNOS-generated NO, and non-adrenergic, non-cholinergic neuronal transmission by nNOS-generated NO would remain unaffected.

Salicylic acid, or 2-hydroxybenzoic acid, is the active COX-1 and COX-2 metabolite of aspirin. Aspirin (acetylsalicylic acid) has the ability to acetylate platelets, and thus is a so called blood thinner, however salicylic acid does not acetylate platelets.

With all pharmaceutical compounds and compositions, the chemical and physical stability of a drug compound is important in the commercial development of that drug substance. Such stability includes the stability at ambient conditions, especially to moisture and under storage conditions. Elevated stability at different conditions of storage is needed to predict the different possible storage conditions during the lifetime of a commercial product. A stable drug avoids the use of special storage conditions as well as frequent inventory replacement. A drug compound must also be stable during the manufacturing process which often requires milling of the drug to achieve drug material with uniform particle size and surface area. Unstable materials often undergo polymorphic changes. Therefore, any modification of a drug substance which enhances its stability profile provides a meaningful benefit over less stable substances.

Several inhibitors of iNOS have been described, such as, for example, S-[2-[(1-iminoethyl)amino]ethyl]-2-methyl-L-cysteine, which is described and claimed in commonly assigned U.S. Pat. No. 6,403,830. That compound, however, is an amorphous solid. It would be desirable, therefore, to provide a crystalline solid form of an iNOS inhibitor such as S-[2-[(1-Iminoethyl)amino]ethyl]-2-methyl-L-cysteine.

### BRIEF DESCRIPTION OF THE DRAWINGS

FIG. 1 is a schematic of S-[2-[(1-Iminoethyl)amino]ethyl]-2-methyl-L-cysteine titration curve, showing all ionization states;

FIG. 2 is a graphical representation of titration curves of S-[2-[(1-Iminoethyl)amino]ethyl]-2-methyl-L-cysteine in water with IRA-400(OH) anion exchange resin. Diamond is pH and square (dashed line) is S-[2-[(1-Iminoethyl)amino]ethyl]-2-methyl-L-cysteine (% initial, by ion chromatography);

FIG. 3 is a graphical representation of titration curves of S-[2-[(1-Iminoethyl)amino]ethyl]-2-methyl-L-cysteine in water with IRA-400(OH) anion exchange resin. Diamond is pH and triangle (broken line) is chloride (by ion chromatography);

FIG. 4 Shows titration curves of S-[2-[(1-Iminoethyl)amino]ethyl]-2-methyl-L-cysteine in water with IRA-400 anion exchange resin;

FIG. 5 shows the relevant binding data associated with increasing pH of the zwitterion of S-[2-[(1-Iminoethyl)amino]ethyl]-2-methyl-L-cysteine;

FIG. 6 is an x-ray powder pattern of S-[2-[(1-Iminoethyl)amino]ethyl]-2-methyl-L-cysteine salicylate monohydrate (Example 10);

FIG. 7 is a graph of differential scanning calorimetry of S-[2-[(1-Iminoethyl)amino]ethyl]-2-methyl-L-cysteine salicylate monohydrate (Example 10);

FIG. 8 is a plot of Thermogravimetric Analysis (TGA) and Scanning Differential Thermal Analysis (SDTA) of S-[2-[(1-Iminoethyl)amino]ethyl]-2-methyl-L-cysteine salicylate monohydrate (Example 10);

FIG. 9 is a graph of a moisture balance plotting relative humidity versus mass change over time;

FIG. 10 is a graph of a moisture balance plotting relative humidity versus mass change and solvent loss over time;

FIG. 11 is an isotherm plot of S-[2-[(1-Iminoethyl)amino]ethyl]-2-methyl-L-cysteine salicylate monohydrate; and

FIG. 12 is the Raman spectrum of S-[2-[(1-Iminoethyl)amino]ethyl]-2-methyl-L-cysteine salicylate monohydrate.

### SUMMARY OF THE INVENTION

The present invention is directed to a novel crystalline salt of S-[2-[(1-Iminoethyl)amino]ethyl]-2-methyl-L-cysteine, pharmaceutical compositions, a process for preparing the novel salt compounds, a process for preparing pharmaceutical compositions, and methods of using said novel salt compound and compositions for inhibiting or modulating nitric oxide synthesis in a subject in need of such inhibition or modulation by administering a salt of a compound which preferentially inhibits or modulates the inducible isoform of nitric oxide synthase over the constitutive isoforms of nitric oxide synthase. The present salt compound possesses useful nitric oxide synthase inhibiting activity, and is expected to be useful in the treatment or prophylaxis of a disease or condition in which the synthesis or oversynthesis of nitric oxide forms a contributory part.

Stoichiometrically, the novel salt is one molecule of S-[2-[(1-Iminoethyl)amino]ethyl]-2-methyl-L-cysteine and one molecule of salicylic acid.

The novel salt is characterized by some or all of the following physical measurements: elemental analysis (such as by combustion analysis), melting point and heat of fusion (differential scanning calorimetry and thermogravimetric analysis), refractive indices (polarized light microscopy), x-ray powder diffraction pattern, moisture sorption (for example, DVS moisture balance) and vibrational signature (Raman spectrum). In another embodiment of the present invention, a method of making S-[2-[(1-Iminoethyl)amino]ethyl]-2-methyl-L-cysteine salicylate monohydrate comprising:
obtaining S-[₂-[(1-Iminoethyl)amino]ethyl]-2-methyl-L-cysteine zwitterion;
adding the S-[2-[(1-Iminoethyl)amino]ethyl]-2-methyl-L-cysteine zwitterion to an appropriate solvent;
adding a salicylic acid to the S-[2-[(1-Iminoethyl)amino]ethyl]-2-methyl-L-cysteine and solvent; and
adding an antisolvent to precipitate S-[2-[(1-Iminoethyl)amino]ethyl]-2-methyl-L-cysteine salicylate monohydrate crystals is disclosed. The solvent may be any appropriate solvent, such as water, dimethylformamide, or mixtures thereof, for example. The antisolvent may be any appropriate antisolvent, such as acetonitrile, methanol, or ethanol, or mixtures thereof, for example.

The present novel crystalline salt can be used to treat diseases involving cartilage degeneration, which takes place in certain conditions such as arthritis. Accordingly, conditions in which there is an advantage in inhibiting NO production from L-arginine include arthritic conditions such as rheumatoid arthritis, osteoarthritis, gouty arthritis, juvenile arthritis, septic arthritis, spondyloarthritis, acute rheumatic arthritis, enteropathic arthritis, neuropathic arthritis, and pyogenic arthritis. In addition, NO-induced depression of chondrocyte respiration could modulate matrix loss and secondary cartilage mineralization in arthritis, in particular osteoarthritis.

Other conditions for which the present crystalline salt may be useful include chronic or inflammatory bowel disease, cardiovascular ischemia, diabetes, congestive heart failure, myocarditis, atherosclerosis, migraine, glaucoma, aortic aneurysm, reflux esophagitis, diarrhea, irritable bowel syndrome, cystic fibrosis, emphysema, asthma, bronchiectasis, hyperalgesia, cerebral ischemia, thrombotic stroke, global ischemia (secondary to cardiac arrest), multiple sclerosis and other central nervous system disorders mediated by NO, for example Parkinson's disease and Alzheimer's disease. Further neurodegenerative disorders in which NO inhibition may be useful include nerve degeneration and/or nerve necrosis in disorders such as hypoxia, hypoglycemia, epilepsy, and in external wounds (such as spinal cord and head injury), hyperbaric oxygen convulsions and toxicity, dementia e.g. pre-senile dementia, and AIDS-related dementia, Sydenham's chorea, Huntington's disease, Amyotrophic Lateral Sclerosis, Korsakoff's disease, imbecility relating to a cerebral vessel disorder, sleeping disorders, schizophrenia, depression, depression or other symptoms associated with Premenstrual Syndrome (PMS), anxiety and septic shock.

The present salt may also be used where nitric oxide inhibition may also play a role in the treatment, such as pain including somatogenic (either nociceptive or neuropathic), both acute and chronic. The present compounds could be used in any situation that a common NSAID or opioid analgesic would traditionally be administered.

Still, other disorders that may be treated by inhibiting NO production with the present salt include opiate tolerance in patients needing protracted opiate analgesics, and benzodiazepine tolerance in patients taking benzodiazepines, and other addictive behavior, for example, nicotine and eating disorders. The present compounds may also be useful as antibacterial agents.

Further conditions in which the present salt may be used to inhibit NO production from L-arginine include systemic hypotension associated with septic and/or toxic shock induced by a wide variety of agents; therapy with cytokines such as TNF, IL-1 and IL-2; and as an adjuvant to short term immunosuppression in transplant therapy.

The present salt may also be useful in the treatment of ocular conditions (such as ocular hypertension retinitis uveitis), systemic lupus erythematosis (SLE), glomerulonephritis, restenosis, inflammatory sequelae of viral infections, acute respiratory distress syndrome (ARDS), oxidant-induced lung injury, IL2 therapy such as in a cancer patient, cachexia, immunosuppression such as in transplant therapy, disorders of gastrointestinal motility, sunburn, eczema, psoriasis, gingivitis, pancreatitis, damage to the gastrointestinal tract resulting from infections, cystic fibrosis, treatment to a dysfunctional immune system such as an adjuvant to short term immunosuppression in organ transplant therapy, induction of labor, adenomatous polyposis, controlling tumor growth, chemotherapy, chemoprevention and bronchitis.

The present invention is also directed to pharmaceutical compositions for the treatment of pain, asthma and other airway disorders, cancer, arthritis, ocular disorders including retinopathies and glaucoma, inflammation related disorders including irritable bowel syndrome, and other disorders in which an excessive production of nitric oxide plays a role, which comprises a therapeutically effective amount of crystalline of S-[2-[(1-Iminoethyl)amino]ethyl]-2-methyl-L-cysteine salicylate monohydrate together with a pharmaceutically acceptable carrier, diluent or vehicle.

Besides being useful for human treatment, this form is also useful for veterinary treatment of companion animals, exotic animals and farm animals, including mammals, rodents, and the like. More preferred animals include horses, dogs, and cats.

### DETAILED DESCRIPTION OF THE INVENTION

Definitions

The terms “treat,” “treating” and “treatment,” as used herein includes prophylactic, palliative treatment, or restorative treatment.

The term “effective amount” means a dose conducive to treatment. An effective amount may be administered in a single dose, or in divided doses over a period of time.

The term “ACE” means acetone.

The term “ACN” means acetonitrile.

The term “amorphous” as applied to S-[2-[(1-Iminoethyl)amino]ethyl]-2-methyl-L-cysteine herein refers to a solid state wherein the S-[2-[(1-Iminoethyl)amino]ethyl]-2-methyl-L-cysteine molecules are present in a disordered arrangement and do not form a distinguishable crystal lattice or unit cell. When subjected to X-ray powder diffraction, amorphous S-[2-[(1-Iminoethyl)amino]ethyl]-2-methyl-L-cysteine does not produce any characteristic crystalline peaks.

The term “crystalline form” as applied to S-[2-[(1-Iminoethyl)amino]ethyl]-2-methyl-L-cysteine herein refers to a solid state form wherein the S-[2-[(1-Iminoethyl)amino]ethyl]-2-methyl-L-cysteine molecules are arranged to form a distinguishable crystal lattice (i) comprising distinguishable unit cells, and (ii) yielding diffraction peaks when subjected to X-ray radiation.

The term “crystallization” as used herein can refer to crystallization and/or recrystallization depending upon the applicable circumstances relating to preparation of S-[2-[(1-Iminoethyl)amino]ethyl]-2-methyl-L-cysteine starting material.

The term “DMF” means. N,N-dimethylformamide.

The term “D/W/A” refers to a ternary solvent system of N,N-dimethylformamide (DMF), water and acetonitrile.

The term “S-[2-[(1-Iminoethyl)amino]ethyl]-2-methyl-L-cysteine drug substance” as used herein means S-[2-[(1-Iminoethyl)amino]ethyl]-2-methyl-L-cysteine per se as qualified by the context in which the term is used, and can refer to unformulated S-[2-[(1-Iminoethyl)amino]ethyl]-2-methyl-L-cysteine or to S-[2-[(1-Iminoethyl)amino]ethyl]-2-methyl-L-cysteine present as an ingredient of a pharmaceutical composition.

The term “DSC” means differential scanning calorimetry.

The term “DTA” means differential thermal analysis.

The term “SDTA” means simultaneous differential thermal analysis.

The term “HPLC” means high pressure liquid chromatography.

The term “IR” means infrared.

The term “NMR” means nuclear magnetic resonance, and may apply to nuclear magnetic resonance spectroscopy.

The term “ml” means milliliters.

The term “mg” means milligrams.

The term “μg” means micrograms

The term “μl” means microliters.

The term “nucleation,” as used herein, means the formation of crystals in a solution.

The term “Purity” herein, unless otherwise qualified, means the chemical purity of S-[2-[(1-Iminoethyl)amino]ethyl]-2-methyl-L-cysteine according to conventional HPLC assay.

The term “PXRD” means powder X-ray diffraction.

The term “rpm” means revolutions per minute.

The term “seeding,” as used herein, means the addition of crystals to a solution for the purpose of initiating or enhancing nucleation.

The term “TGA” means thermogravimetric analysis.

The term “Tm” means melting temperature.

The term “free zwitterion” means a molecule that carries both a positive and negative charge such that the net charge is zero.

S-[2-[(1-Iminoethyl)amino]ethyl]-2-methyl-L-cysteine salicylate monohydrate crystalline salt will be useful for treating, among other things, inflammation in a subject, or for treating other nitric oxide synthase-mediated disorders, such as, as an analgesic in the treatment of pain and headaches, or as an antipyretic for the treatment of fever. For example, S-[2-[(1-Iminoethyl)amino]ethyl]-2-methyl-L-cysteine salicylate monohydrate crystalline salt will be useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. Conditions in which the S-[2-[(1-Iminoethyl)amino]ethyl]-2-methyl-L-cysteine salicylate monohydrate crystalline salt will provide an advantage in inhibiting NO production from L-arginine include arthritic conditions.

S-[2-[(1-Iminoethyl)amino]ethyl]-2-methyl-L-cysteine salicylate monohydrate crystalline salt will be further useful in the treatment of asthma, bronchitis, menstrual cramps (e.g., dysmenorrhea), premature labor, tendinitis, bursitis, skin-related conditions such as psoriasis, eczema, burns, sunburn, dermatitis, pancreatitis, hepatitis, and from post-operative inflammation including from ophthalmic surgery such as cataract surgery and refractive surgery. S-[2-[(1-Iminoethyl)amino]ethyl]-2-methyl-L-cysteine salicylate monohydrate crystalline salt also would be useful to treat gastrointestinal conditions such as inflammatory bowel disease, Crohn's disease, gastritis, irritable bowel syndrome and ulcerative colitis.

S-[2-[(1-Iminoethyl)amino]ethyl]-2-methyl-L-cysteine salicylate monohydrate crystalline salt would be useful for the prevention or treatment of cancer, such as colorectal cancer, and cancer of the breast, lung, prostate, bladder, cervix and skin. S-[2-[(1-Iminoethyl)amino]ethyl]-2-methyl-L-cysteine salicylate monohydrate crystalline salt of the invention would be useful in treating inflammation and tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like. The S-[2-[(1-Iminoethyl)amino]ethyl]-2-methyl-L-cysteine salicylate monohydrate crystalline salt would also be useful in the treatment of ophthalmic diseases, such as glaucoma, retinitis, retinopathies, uveitis, ocular photophobia, and of inflammation and pain associated with acute injury to the eye tissue. Of particular interest among the uses of the present inventive S-[2-[(1-Iminoethyl)amino]ethyl]-2-methyl-L-cysteine salicylate monohydrate crystalline salt is the treatment of glaucoma, especially where symptoms of glaucoma are caused by the production of nitric oxide, such as in nitric oxide-mediated nerve damage. The S-[2-[(1-Iminoethyl)amino]ethyl]-2-methyl-L-cysteine salicylate monohydrate crystalline salt would also be useful in the treatment of pulmonary inflammation, such as that associated with viral infections and cystic fibrosis. The S-[2-[(1-Iminoethyl)amino]ethyl]-2-methyl-L-cysteine salicylate monohydrate crystalline salt would also be useful for the treatment of certain central nervous system disorders, such as cortical dementias including Alzheimer's disease, and central nervous system damage resulting from stroke, ischemia and trauma. The S-[2-[(1-Iminoethyl)amino]ethyl]-2-methyl-L-cysteine salicylate monohydrate crystalline salt is useful as an anti-inflammatory agent, such as for the treatment of arthritis, with the additional benefit of having significantly less harmful side effects.

S-[2-[(1-Iminoethyl)amino]ethyl]-2-methyl-L-cysteine salicylate monohydrate crystalline salt would also be useful in the treatment of allergic rhinitis, respiratory distress syndrome, endotoxin shock syndrome, and atherosclerosis.

S-[2-[(1-Iminoethyl)amino]ethyl]-2-methyl-L-cysteine salicylate monohydrate crystalline salt would also be useful in the treatment of pain, including but not limited to postoperative pain, dental pain, muscular pain, and pain resulting from cancer. S-[2-[(1-Iminoethyl)amino]ethyl]-2-methyl-L-cysteine salicylate monohydrate crystalline salt would be useful for the prevention of dementias, such as Alzheimer's disease.

Besides being useful for human treatment, S-[2-[(1-Iminoethyl)amino]ethyl]-2-methyl-L-cysteine salicylate monohydrate crystalline salt is also useful for veterinary treatment of companion animals, exotic animals and farm animals, including mammals, rodents, and the like. More preferred animals include horses, dogs, and cats.

The present S-[2-[(1-Iminoethyl)amino]ethyl]-2-methyl-L-cysteine salicylate monohydrate crystalline salt may also be used in co-therapies, partially or completely, in place of other conventional antiinflammatory therapies, such as together with steroids, NSAIDs, COX-2 selective inhibitors, 5-lipoxygenase inhibitors, LTB₄ antagonists and LTA₄ hydrolase inhibitors.

Other conditions in which the S-[2-[(1-Iminoethyl)amino]ethyl]-2-methyl-L-cysteine salicylate monohydrate crystalline salt of the present invention will provide an advantage in inhibiting NO inhibition include cardiovascular ischemia, diabetes (type I or type II), congestive heart failure, myocarditis, atherosclerosis, migraine, glaucoma, aortic aneurysm, reflux esophagitis, diarrhea, irritable bowel syndrome, cystic fibrosis, emphysema, asthma, bronchiectasis, hyperalgesia (allodynia), cerebral ischemia (both focal ischemia, thrombotic stroke and global ischemia (for example, secondary to cardiac arrest), multiple sclerosis and other central nervous system disorders mediated by NO, for example Parkinson's disease. Further neurodegenerative disorders in which NO inhibition may be useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia e.g. pre-senile dementia, and AIDS-related dementia, cachexia, Sydenham's chorea, Huntington's disease, Amyotrophic Lateral Sclerosis, Korsakoff's disease, imbecility relating to a cerebral vessel disorder, sleeping disorders, schizophrenia, depression, depression or other symptoms associated with Premenstrual Syndrome (PMS), anxiety and septic shock.

The S-[2-[(1-Iminoethyl)amino]ethyl]-2-methyl-L-cysteine salicylate monohydrate crystalline salt of the present invention will also be useful in the treatment of pain including somatogenic (either nociceptive or neuropathic), both acute and chronic. S-[2-[(1-Iminoethyl)amino]ethyl]-2-methyl-L-cysteine salicylate monohydrate crystalline salt could be used in any situation including neuropathic pain that a common NSAID or opioid analgesic would traditionally be administered.

Still other disorders or conditions which will be advantageously treated by the S-[2-[(1-Iminoethyl)amino]ethyl]-2-methyl-L-cysteine salicylate monohydrate crystalline salt of the present invention include treatment or prevention of opiate tolerance in patients needing protracted opiate analgesics, and benzodiazepine tolerance in patients taking benzodiazepines, and other addictive behavior, for example, nicotine addiction, alcoholism, and eating disorders.

The S-[2-[(1-Iminoethyl)amino]ethyl]-2-methyl-L-cysteine salicylate monohydrate crystalline salt of the present invention will also be useful in the treatment or prevention of drug withdrawal symptoms, for example treatment or prevention of symptoms of withdrawal from opiate, alcohol, or tobacco addiction.

The S-[2-[(1-Iminoethyl)amino]ethyl]-2-methyl-L-cysteine salicylate monohydrate crystalline salt may also be useful to prevent tissue damage when therapeutically combined with antibacterial or antiviral agents.

The S-[2-[(1-Iminoethyl)amino]ethyl]-2-methyl-L-cysteine salicylate monohydrate crystalline salt of the present invention will also be useful in inhibiting NO production from L-arginine including systemic hypotension associated with septic and/or toxic hemorrhagic shock induced by a wide variety of agents; therapy with cytokines such as TNF, IL-1 and IL-2; and as an adjuvant to short term immunosuppression in transplant therapy.

The present invention is further directed to the use of the S-[2-[(1-Iminoethyl)amino]ethyl]-2-methyl-L-cysteine salicylate monohydrate crystalline salt of the present invention for the treatment and prevention of neoplasias. The neoplasias that will be treatable or preventable by the S-[2-[(1-Iminoethyl)amino]ethyl]-2-methyl-L-cysteine salicylate monohydrate crystalline salt and methods of the present invention include brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. Preferably, the neoplasia to be treated is selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present S-[2-[(1-Iminoethyl)amino]ethyl]-2-methyl-L-cysteine salicylate monohydrate crystalline salt and methods can also be used to treat the fibrosis which occurs with radiation therapy. The present S-[2-[(1-Iminoethyl)amino]ethyl]-2-methyl-L-cysteine salicylate monohydrate crystalline salt and methods can be used to treat subjects having adenomatous polyps, including those with familial adenomatous polyposis (FAP). Additionally, the present S-[2-[(1-Iminoethyl)amino]ethyl]-2-methyl-L-cysteine salicylate monohydrate crystalline salt and methods can be used to prevent polyps from forming in patients at risk of FAP.

Conjunctive treatment of a S-[2-[(1-Iminoethyl)amino]ethyl]-2-methyl-L-cysteine salicylate monohydrate crystalline salt of the present invention with another antineoplastic agent will produce a synergistic effect or alternatively reduce the toxic side effects associated with chemotherapy by reducing the therapeutic dose of the side effect-causing agent needed for therapeutic efficacy or by directly reducing symptoms of toxic side effects caused by the side effect-causing agent. S-[2-[(1-Iminoethyl)amino]ethyl]-2-methyl-L-cysteine salicylate monohydrate crystalline salt of the present invention will further be useful as an adjunct to radiation therapy to reduce side effects or enhance efficacy.

In the present invention, another agent which can be combined therapeutically with the S-[2-[(1-Iminoethyl)amino]ethyl]-2-methyl-L-cysteine salicylate monohydrate crystalline salt of the present invention includes any therapeutic agent which is capable of inhibiting the enzyme cyclooxygenase-2 (“COX-2”). Preferably such COX-2 inhibiting agents inhibit COX-2 selectively relative to the enzyme cyclooxygenase-1 (“COX-1”). Such a COX-2 inhibitor is known as a “COX-2 selective inhibitor”. COX-2 selective inhibitors useful in therapeutic combination with the S-[2-[(1-Iminoethyl)amino]ethyl]-2-methyl-L-cysteine salicylate monohydrate crystalline salt of the present invention include celecoxib, valdecoxib, deracoxib, etoricoxib, rofecoxib, ABT-963 (2-(3,4-difluorophenyl)-4-(3-hydroxy-3-methyl-1-butoxy)-5-[4-(methylsulfonyl)phenyl-3(2H)-pyridazinone; described in PCT Patent Application No. WO 00/24719), or meloxicam. S-[2-[(1-Iminoethyl)amino]ethyl]-2-methyl-L-cysteine salicylate monohydrate crystalline salt of the present invention can also be advantageously used in therapeutic combination with a prodrug of a COX-2 selective inhibitor, for example parecoxib.

Another chemotherapeutic agent which will be useful in combination with the S-[2-[(1-Iminoethyl)amino]ethyl]-2-methyl-L-cysteine salicylate monohydrate crystalline salt of the present invention can be selected, for example, from the following non-comprehensive and non-limiting list:

Alpha-difluoromethylornithine (DFMO), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT, uricytin, Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin, trimelamol, Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605 Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 zorubicin, alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristo-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemex CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, Tsumura EPMTC, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, Yamanouchi YM-534, uroguanylin, combretastatin, dolastatin, idarubicin, epirubicin, estramustine, cyclophosphamide, 9-amino-2-(S)-camptothecin, topotecan, irinotecan (Camptosar), exemestane, decapeptyl (tryptorelin), or an omega-3 fatty acid.

Examples of radioprotective agents which may be used in a combination therapy with the S-[2-[(1-Iminoethyl)amino]ethyl]-2-methyl-L-cysteine salicylate monohydrate crystalline salt of this invention include AD-5, adchnon, amifostine analogues, detox, dimesna, 1-102, MM-159, N-acylated-dehydroalanines, TGF-Genentech, tiprotimod, amifostine, WR-151327, FUT-187, ketoprofen transdermal, nabumetone, superoxide dismutase (Chiron) and superoxide dismutase Enzon.

The S-[2-[(1-Iminoethyl)amino]ethyl]-2-methyl-L-cysteine salicylate monohydrate crystalline salt of the present invention will also be useful in treatment or prevention of angiogenesis-related disorders or conditions, for example, tumor growth, metastasis, macular degeneration, and atherosclerosis.

In a further embodiment, the present invention also provides therapeutic combinations for the treatment or prevention of ophthalmic disorders or conditions such as glaucoma. For example the present inventive S-[2-[(1-Iminoethyl)amino]ethyl]-2-methyl-L-cysteine salicylate monohydrate crystalline salt advantageously will be used in therapeutic combination with a drug which reduces the intraocular pressure of patients afflicted with glaucoma. Such intraocular pressure-reducing drugs include without limitation latanoprost, travoprost, bimatoprost, or unoprostol. The therapeutic combination of the S-[2-[(1-Iminoethyl)amino]ethyl]-2-methyl-L-cysteine salicylate monohydrate crystalline salt of the present invention plus an intraocular pressure-reducing drug will be useful because each is believed to achieve its effects by affecting a different mechanism.

In another combination of the present invention, the present inventive S-[2-[(1-Iminoethyl)amino]ethyl]-2-methyl-L-cysteine salicylate monohydrate crystalline salt can be used in therapeutic combination with an antihyperlipidemic or cholesterol-lowering drug such as a benzothiepine or a benzothiazepine antihyperlipidemic drug. Examples of benzothiepine antihyperlipidemic drugs useful in the present inventive therapeutic combination can be found in U.S. Pat. No. 5,994,391, herein incorporated by reference. Some benzothiazepine antihyperlipidemic drugs are described in WO 93/16055. Alternatively, the antihyperlipidemic or cholesterol-lowering drug useful in combination with a compound of the present invention can be an HMG Co-A reductase inhibitor. Examples of HMG Co-A reductase inhibitors useful in the present therapeutic combination include, individually, benfluorex, fluvastatin, lovastatin, provastatin, simvastatin, atorvastatin, cerivastatin, bervastatin, ZD-9720 (described in PCT Patent Application No. WO 97/06802), ZD-4522 (CAS No. 147098-20-2 for the calcium salt; CAS No. 147098-18-8 for the sodium salt; described in European Patent No. EP 521471), BMS 180431 (CAS No. 129829-03-4), or NK-104 (CAS No. 141750-63-2). The therapeutic combination of the S-[2-[(1-Iminoethyl)amino]ethyl]-2-methyl-L-cysteine salicylate monohydrate crystalline salt of the present invention plus an antihyperlipidemic or cholesterol-lowering drug will be useful, for example, in reducing the risk of formation of atherosclerotic lesions in blood vessels. For example, atherosclerotic lesions often initiate at inflamed sites in blood vessels. It is established that antihyperlipidemic or cholesterol-lowering drug reduce risk of formation of atherosclerotic lesions by lowering lipid levels in blood. Without limiting the invention to a single mechanism of action, it is believed that one way the S-[2-[(1-Iminoethyl)amino]ethyl]-2-methyl-L-cysteine salicylate monohydrate crystalline salt of the present combination will work in concert to provide improved control of atherosclerotic lesions by, for example, reducing inflammation of the blood vessels in concert with lowering blood lipid levels.

In another embodiment of the invention, the present S-[2-[(1-Iminoethyl)amino]ethyl]-2-methyl-L-cysteine salicylate monohydrate crystalline salt can be used in combination with other compounds or therapies for the treatment of central nervous conditions or disorders such as migraine. For example, the present S-[2-[(1-Iminoethyl)amino]ethyl]-2-methyl-L-cysteine salicylate monohydrate crystalline salt can be used in therapeutic combination with caffeine, a 5-HT-1B/1D agonist (for example, a triptan such as sumatriptan, naratriptan, zolmitriptan, rizatriptan, almotriptan, or frovatriptan), a dopamine D4 antagonist (e.g., sonepiprazole), aspirin, acetaminophen, ibuprofen, indomethacin, naproxen sodium, isometheptene, dichloralphenazone, butalbital, an ergot alkaloid (e.g., ergotamine, dihydroergotamine, bromocriptine, ergonovine, or methyl ergonovine), a tricyclic antidepressant (e.g., amitriptyline or nortriptyline), a serotonergic antagonist (e.g., methysergide or cyproheptadine), a beta-andrenergic antagonist (e.g., propranolol, timolol, atenolol, nadolol, or metprolol), or a monoamine oxidase inhbitor (e.g., phenelzine or isocarboxazid).

A further embodiment provides a therapeutic combination of the S-[2-[(1-Iminoethyl)amino]ethyl]-2-methyl-L-cysteine salicylate monohydrate crystalline salt of the present invention with an opioid compound. Opioid compounds useful in this combination include without limitation morphine, methadone, hydromorphone, oxymorphone, levorphanol, levallorphan, codeine, dihydrocodeine, dihydrohydroxycodeinone, pentazocine, hydrocodone, oxycodone, nalmefene, etorphine, levorphanol, fentanyl, sufentanil, DAMGO, butorphanol, buprenorphine, naloxone, naltrexone, CTOP, diprenorphine, beta-funaltrexamine, naloxonazine, nalorphine, pentazocine, nalbuphine, naloxone benzoylhydrazone, bremazocine, ethylketocyclazocine, U50,488, U69,593, spiradoline, nor-binaltorphimine, naltrindole, DPDPE, [D-la², glu⁴]deltorphin, DSLET, met-enkephalin, leu-enkaphalin, beta-endorphin, dynorphin A, dynorphin B, and alpha-neoendorphin. An advantage to the combination of the S-[2-[(1-Iminoethyl)amino]ethyl]-2-methyl-L-cysteine salicylate monohydrate crystalline salt of the present invention with an opioid compound is that the present inventive S-[2-[(1-Iminoethyl)amino]ethyl]-2-methyl-L-cysteine salicylate monohydrate crystalline salt will allow a reduction in the dose of the opioid compound, thereby reducing the risk or severity of opioid side effects, such as opioid addiction.

A method to make S-[2-[(1-iminoethyl)amino]ethyl]-2-methyl-L-cysteine dihydrochloride is described in commonly assigned U.S. Pat. No. 6,403,830, incorporated herein by reference.

Briefly, synthesis of S-[2-[(1-iminoethyl)amino]ethyl]-2-methyl-L-cysteine dihydrochloride may be performed as in the following Example 1:

### Example 1

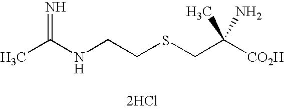

### S-[2-[(1-Iminoethyl)amino]ethyl]-2-methyl-L-cysteine, dihydrochloride

### Example-1A)

### N-Boc-cysteamine

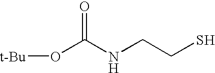

A 3 L 4-neck RB flask was purged with nitrogen for 20 min and then charged sequentially with 2-aminoethanethiol hydrochloride (113.6 g, 1 mol), di-tert-butyl-dicarbonate (218.3 g, 1 mol) and 500 mL of toluene. The mixture was cooled with an ice-water bath and purged with nitrogen for 10 min. Sodium hydroxide (2.5N, 880 mL, 2.2 mol) was added to the stirring mixture in about 1.5 h at between 0 and 11° C. After the addition of sodium hydroxide was complete, the cooling bath was removed and the resulting reaction mixture was allowed to warm up to room temperature and stirred at ambient temperature overnight. This provided a solution of the title product.

### Example-1B)

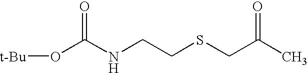

The product solution of Example-1A was cooled with an ice-water bath. A sample of chloroacetone (101.8 g, 1.1 mol) was added to the vigorously stirred reaction mixture over about 50 min at between 8 and 11° C. After the addition of chloroacetone was completed, the cooling bath was removed and the resulting reaction mixture was allowed to stir at room temperature overnight. The toluene layer was separated, washed with water (250 mL) and concentrated on a rotary evaporator at 85° C. under house vacuum followed by high vacuum to give the crude titled compound (225.7 g, 96.7%). ¹H NMR (CDCl₃, 400 MHz) δ 4.95 (bs, 1H), 3.20 (m, 4H), 2.54 (t, 2H), 2.20 (s, 3H), 1.35 (s, 9H).

### Example-1C)

### [2-[[(4-Methyl-2,5-dioxo-4-imidazolidinyl)methyl]thio]ethyl]carbamic acid, 1,1-dimethylethyl ester

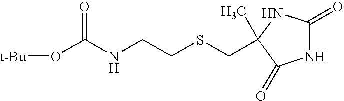

To a 3 L 4-neck RB flask equipped with an overhead stirrer, a thermocouple and a condenser connected to an empty flask and a caustic trap, was added the product of Example-1B (70 g, 0.3 mol), absolute ethanol (80 mL), sodium cyanide (19.1 g, 0.39 mol), ammonium carbonate (43.3 g, 0.45 mol) and water (720 mL) in this order. The 4^{th} neck was closed with a stopper. The resulting reaction mixture was heated at between 67 and 68° C. for 6 h. Subsequently, the almost clear brown solution was cooled to room temperature. Upon cooling, solid began to form and the heterogeneous mixture was stirred at room temperature overnight. The reaction mixture was then acidified with 12% hydrochloric acid to pH 2 in about 1 h at between −2 and 2° C. The cold reaction mixture was stirred at pH2 for additional 30 min and then filtered. The flask was rinsed with distilled water (2×250 mL) and each rinse was used to wash the solid cake. The solid was again washed with distilled water (2×250 mL) and then air-dried for 4 days. The dry solid was triturated with 200 mL of toluene for 0.5 h. The slurry was filtered. The solid was rinsed sequentially with toluene (50 mL) and 1:4 ratio of toluene/hexane (100 mL) and then air-dried at room temperature overnight to give 83.1% yield of the titled compound, m.p. 134-136° C. ¹H NMR (DMSO_{d6}, 400 MHz) δ 10.62 (s, 1H), 7.85 (s, 1H), 6.83 (m, 0.9H), 6.48 (bs, 0.1H), 3.29 (s, 2H), 2.99 (m, 2H), 2.71 (s, 2H), 2.95 (m, 2H), 1.32 (s, 9H), 1.24 (s, 3H); ¹³C NMR (DMSO_{d6}, 400 MHz), δ 178.1, 157.1, 156.1, 78.4, 63.7, 40.7, 39.4, 33.2, 28.9, 23.8.

Analysis Calcd for C₁₂H₂₁N₃O₄S: C, 47.51; H, 6.98; N, 13.85; S, 10.57. Found: C, 47.76; H, 6.88; N, 13.77; S, 10.75.

### Example-1D)

### R and S-[2-[[(4-Methyl-2,5-dioxo-4-imidazolidinyl)methyl]thio]ethyl]carbamic acid, 1,1-dimethylethyl ester

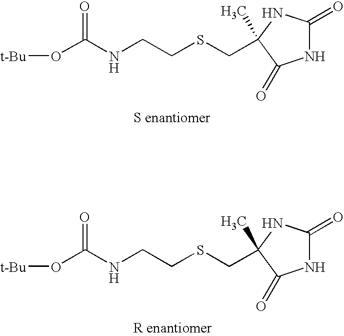

The reaction product of Example-1C was separated into its R and S enantiomers on a Chiralpak® AD column eluting with methanol. The S isomer was the first eluting isomer followed by its R enantiomer. Both isomers were used in subsequent transformations. S enantiomer:

[α]in MeOH at 25° C.=+43.0 (365 nm). ¹HNMR: (400 mHz, CD₃OD) δ 1.49 (s, 9H), 2.05 (s, 3H), 2.65 (t, 2H), 2.9 (q, 2H, d), 3.20 (m, 2H). IR: λcm⁻¹=1772, 1709.

Analysis calculated for C₁₂H₂₁N₃O₄S (formula weight=303.38): C 47.51, H 6.98, N 13.85. Found: C, 47.39; H, 6.62; N, 13.83. M+H=304.

R Enantiomer:

[α] in MeOH at 25° C.=−46.3 (365 nm). ¹HNMR: (400 mHz, CD₃OD) δ 1.48 (s, 9H), 2.05 (s, 3H), 2.65 (t, 2H), 2.85 (q, 2H, d), 3.18 (m, 2H). IR: λcm⁻¹=1770, 1711.

Analysis calculated for C₁₂H₂₁N₃O₄S (formula weight=303.38): C, 47.51; H, 6.98; N, 13.85. Found: C, 48.15; H, 7.04; N, 14.37. M+H=304.

### Example-1E)

### S-(2-aminoethyl)-2-methyl-L-cysteine

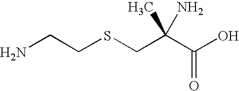 Acid Hydrolysis Method:

A 500 mL three-necked round bottom flask equipped with a distillation condenser was charged with the R-isomer product of Example-1D (45.8 g, 150.9 mmol) and treated portion wise with 48% aq. HBr (160 mL) at room temperature with stirring. After the gas evolution ceased, the mixture was heated with a beating mantle until the pot temperature reached to 126° C. while the volatile t-butyl bromide (bp 72-74° C.) followed by a small amount of aq. HBr (approx. 15 mL) were distilled off. The distillation condenser was replaced with a reflux condenser and the mixture was heated at reflux for 30 hours. The solution was concentrated and the residue was dissolved in water (250 mL) and loaded on to a Dowex® 50WX4-200 ion-exchange resin (8.5×11 cm) and eluted with water (2 L) followed by dilute aqueous ammonium hydroxide (30 mL of 28-30% ammonium hydroxide diluted to 1000 mL with water, 3 L). Fractions containing the desired product were combined, concentrated, and dried under vacuum at 75-80° C. for two hours to give 22.1 g (82%) of the title product, S-(2-aminoethyl)-2-methyl-L-cysteine, as a white solid. Proton and C-13 NMR spectra are consistent with the title product. Mp 157° C. ¹H NMR (400 MHz, D₂O) δ 1.19 (3H, s), 2.53 (1H, d, J=13.6 Hz), 2.57-2.72 (2H, m), 2.92 (1H, d, J=13.6 Hz), 2.92 (2H, t, J=6.8 Hz); ¹³C NMR (100 MHz, D₂O) δ 24.7, 31.3, 38.9, 40.9, 59.6, 180.7.

Analysis Calculated for C₆H₁₄N₂O₂S+0.1H₂O: C, 40.02; H, 7.95; N, 15.56; S, 17.81. Found: C, 39.93; H, 7.98; N, 15.38; S, 17.70.

Base Hydrolysis Method:

To a stainless steel autoclave equipped with agitation was added 24.2 g (0.08 moles) of the R-isomer product of Example-1D. After purging the apparatus with nitrogen, 128 g (0.32 moles) of 10% caustic was added generating a solution. The autoclave was sealed and heated to 120° C. for 30 hours. After cooling to room temperature, the autoclave was vented to give 142 ml (151 g) of an aqueous solution of the sodium salt of the title product. H¹NMR (sample acidified with HCl and diluted with D₂O, 400 MHz): δ 1.47 (s, 3H), 2.75 (m, 2H), 2.90 (d, 1H, J=14.8 Hz), 3.06 (t, 2H, J=6.4 Hz), 3.14 (d, 1H, J=14.8 Hz). C¹³NMR (sample acidified with HCl and diluted with D₂O, 100 MHz): δ 172.9, 60.8, 39.1, 39.0, 30.4, 22.2. MS (MS/CI-LC) M+1 179.

DBU (218 μL; 1.46 mmol) and ethyl acetimidate hydrochloride (171 mg; 1.34 mmol) were dissolved in ethanol (6 mL) in a 25 mL, one-necked, round-bottomed flask at room temperature (˜20° C.). The title product of Example-1E (200 mg; 1.12 mmol) was added in one portion to this solution. The mixture was stirred until the title product of Example-1E was consumed (1-2 hours). The mixture was chilled with an ice-bath and then treated with 6 M HCl (830 μL). ¹HNMR analysis indicated a chemical yield of 95 mole % or better. The solvent was evaporated and the title product of Example-I was purified by reverse-phase or ion-exchange chromatography.

A 210 gm solution (containing ˜20 g of the title product of Example-1E of the base hydrolysis reaction product was put into a 500 mL, three-necked, round-bottomed flask. The apparatus was equipped with a mechanical stirrer, a Dean-Stark apparatus (20 mL with a stopcock), a condenser, and a temperature controller. Water (140 mL) was distilled off from the mixture. 1-butanol (150 mL) was added to the pot and the remaining water (37 mL) was distilled azeotropically. Additional 1-butanol (13 mL) was removed by distillation until the pot temperature reached 117° C. The butanol slurry was cooled to room temperature and filtered through a pad of celite. The salts were washed with 1-butanol (2×20 mL). DBU (21.8 μL; 146 mmol) and ethyl acetimidate hydrochloride (17.1 mg; 134 mmol) were dissolved in 1-butanol (40 mL) in a 500 mL, three-necked, round-bottomed flask at room temperature. The apparatus was equipped with a mechanical stirrer, an addition funnel, and a temperature probe. The title product of Example-1E/1-butanol solution was put into the addition funnel and added to the ethyl acetimidate/DBU solution while maintaining the pot temperature below 25° C. The mixture was stirred until the starting material was consumed (2-3 hours). A solution of conc. HCl (94 mL) and water (100 mL) was put into a 1 L, three-necked, round-bottomed flask and chilled to 0° C. The apparatus was equipped with a mechanical stirrer, an addition funnel, and a temperature probe. The reaction mixture was put into the addition funnel. The reaction mixture was added to the aqueous HCl solution while maintaining the temperature below 25° C. Ethyl acetate (100 mL) was added to the solution and the layers were separated. The aqueous layer was washed once more with ethyl acetate (100 mL). ¹HNMR analysis indicated a chemical yield of 95 mole % or better. This title product of Example-1 was purified by reverse-phase, ion-exchange chromatography, hydrophobic interaction chromatography, or combination thereof. ¹HNMR (400 MHz, D₂O) δ 1.49 (3H, s), 2.08 (3H, s), 2.74 (2H, m), 2.91 (1H, d), 3.17 (1H, d), 3.35 (2H, t).

### Example 2

### Preparation of the Zwitterion

In an embodiment of the present invention, excess acid may be removed from the S-[2-[(1-Iminoethyl)amino]ethyl]-2-methyl-L-cysteine dihydrochloride concentrate using anion exchange resin. It was additionally discovered that the monohydrochloro, free zwitterion, or other fractional acid derivative of S-[2-[(1-Iminoethyl)amino]ethyl]-2-methyl-L-cysteine could be prepared using the anion exchange resin. The anion exchange method is preferred for preparing the monohydrochloride and the free zwitterion due to its simplicity. S-[2-[(1-Iminoethyl)amino]ethyl]-2-methyl-L-cysteine with less than 0.5 equivalents of acid and low excess salt is especially useful for pharmaceutical preparation of alternative salt forms.

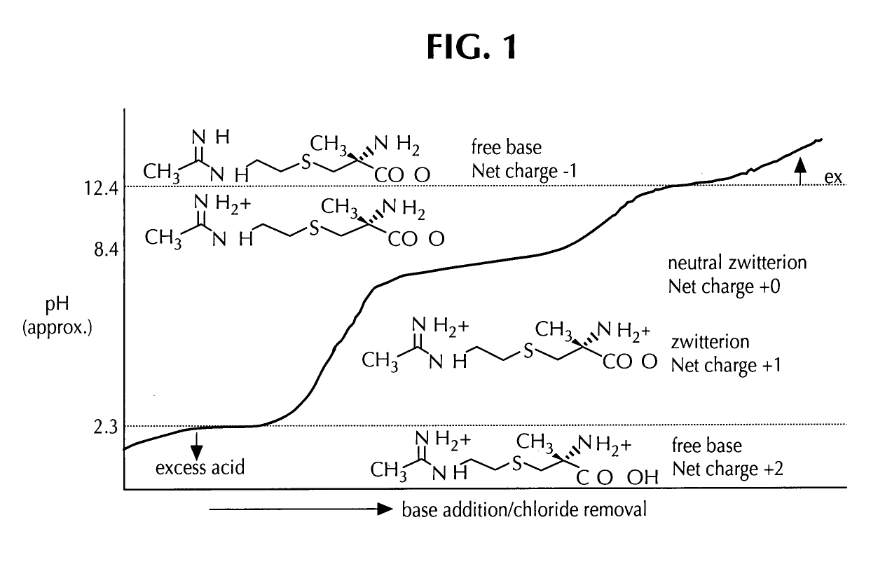

FIG. 1 shows a schematic representation of the compound titration curve. The parent S-[2-[(1-Iminoethyl)amino]ethyl]-2-methyl-L-cysteine molecule has 3 ionizable groups and can exist in 4 ionization states.

At low pH, the molecule exists as a +2 charged free acid, with the carboxylic acid, amine and amidine moieties protonated. This is the ionization state for the dihydrochloride salt.

As the pH is increased, the carboxylic acid group is the first group to deprotonate, and this produces a net charge on the molecule of +1. If the pH increase is generated by addition of sodium hydroxide to S-[2-[(1-Iminoethyl)amino]ethyl]-2-methyl-L-cysteine, the sodium dihydrochloride salt is formed. Other bases would make their corresponding salt forms. If the increase in pH is due to removal of chloride ions by anion exchange processing, the product is the monohydrochloride salt with no sodium or other counterions.

As the pH is further increased, the amine group deprotonates (about pKa=8.4) producing the neutral zwitterionic form of the molecule. A positive charge still resides on the amidine, and a negative charge still resides on the carboxyl group. In contrast, if such material is made by the addition of sodium hydroxide to the dihydrochloride, the resulting product is the monohydrochloro sodium salt, mixed with one equivalent of sodium chloride. The material prepared by the anion exchange resin approach is the free zwitterion.

Further increases in pH lead to deprotonation of the amidine ion (pKa ˜12.5). The molecule in this pH range is the free base and acid salt. Note that the free base is preferably not prepared by the anion exchange method, since the negatively charged molecule binds with the anion exchange resin.

### Example 3

### Preparation of Free Zwitterion

60 g of Amberlite IRA400 (OH) resin was prewashed with 4.7 percent (by weight) ammonium hydroxide (50 ml of 28 percent ammonium hydroxide, 250 ml deionized water), followed by extensive washing with deionized water. The final conductivity was 6.1 μS.

Samples containing about 0.9 g of S-[2-[(1-Iminoethyl)amino]ethyl]-2-methyl-L-cysteine di-hydrochloride in 142 ml HCl/water solution, were concentrated on a rotary evaporator at 60° C. to an oil. To the oil, diluted to 60 ml with deionized water, was added aliquots of 0.5 g of washed anion exchange resin while stirring. At five minutes after each aliquot of resin was added, the solution pH was measured and a sample removed through a syringe filter. A total of 9 g of anion exchange resin was added. The final pH was 10.8. The resin was removed by filtration and the filtrate was concentrated to an oil by rotary evaporation at 60° C.; no solids formed. The starting material, final filtrate and all intermediate samples were assayed by HPLC and ion chromatography for chloride.

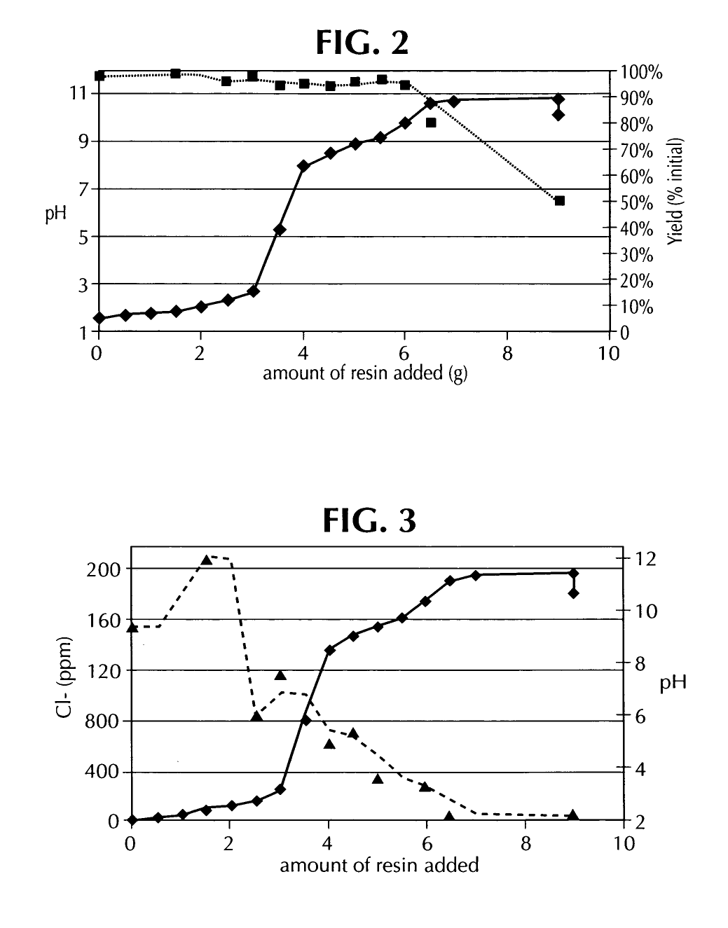

FIG. 2 shows the pseudo-titration curve for S-[2-[(1-Iminoethyl)amino]ethyl]-2-methyl-L-cysteine in water using the anion exchange resin to adjust pH. The diamond (solid line) is pH and square (dashed line) is S-[2-[(1-Iminoethyl)amino]ethyl]-2-methyl-L-cysteine (percent initial, by ion chromatography). FIG. 3 shows the pseudo-titration curve for S-[2-[(1-Iminoethyl)amino]ethyl]-2-methyl-L-cysteine in water using the anion exchange resin to adjust pH. The diamond (solid line) is pH and triangle (broken line) is chloride (by ion chromatography).

These curves are not true titration curves since samples were withdrawn during the progress of the reaction, and since true equilibrium was not attained before the increments of resin were added. Nevertheless, the graphs of FIG. 2 and FIG. 3 illustrate the expected trends. As resin is added to the S-[2-[(1-Iminoethyl)amino]ethyl]-2-methyl-L-cysteine solution the pH rises with change in slope around pH's of 2, 9 and 11. The pH's of slower rise are representative of the pK's of the carboxylic acid, amine and amidine functional groups, respectively. Above a pH of 10, the S-[2-[(1-Iminoethyl)amino]ethyl]-2-methyl-L-cysteine concentration in solution decreases. At this point, the S-[2-[(1-Iminoethyl)amino]ethyl]-2 methyl-L-cysteine is gaining a net negative charge and is binding to the resin. The chloride results show some variation between samples but in general show the trend of decreasing chloride content with increasing pH. The final chloride content is approximately 0.04 mol equivalents. HPLC assay of the samples showed no degradation.

### Example 4

### Removal of Excess HCl to Adjust Acid Equivalents

To 3.3 g of sample containing around 305 mg/ml S-[2-[(1-Iminoethyl)amino]ethyl]-2-methyl-L-cysteine dihydrochloride and 0.23 eq excess HCl, was added 16.7 g of deioinized water. The pH was 1.04. To 14 ml of this solution, prewashed Amberlite 400(OH) resin was added to obtain a pH of 2.5. The anion exchange treatment lightened the color of the solution from light yellow to water white. The resin was removed by filtration and the starting material and filtrate product were analyzed by chloride titration and HPLC.

Qualitative analysis of the starting material and product by HPLC found no new peaks and no increase in impurities. The results from chloride analysis by titration show that the chloride was reduced from 2.18 equivalents to around 1.14 equivalents. Although not demonstrated here, the chloride could be adjusted to the desired target by addition of HCl.

### Example 5

### Preparation of Free Zwitterion

3.3 g of a sample containing about 1 g of S-[2-[(1-Iminoethyl)amino]ethyl]-2-methyl-L-cysteine di-hydrochloride was diluted to 20 g. Aliquots of prewashed Amberlite IRA400 (OH) resin was added to the solution and samples were periodically withdrawn through a syringe filter. Intermediate resin filtrations were performed at pH of 7.1 and 8.8 by filtering off the resin in solution and then continuing to add fresh resin to the filtrate. This was done to drive the chloride removal equilibrium and minimize product adsorption. After the final pH of 11.2 was attained, the resin was filtered off. The starting materials, intermediate samples and final filtrate were analyzed.

The resulting samples were analyzed by HPLC. No difference was seen between the HPLC traces of the starting material and product at pH of 11 within a few hours. However, some degradation peaks at around 2-3 peak area % were seen in the high pH samples after storage at room temperature for around 10 days.

### Example 6

### Preparation of Free Zwitterion

Amberlite IRA400 (Cl) resin was rinsed with 3M HCl, water, and then 3M NaOH. Aliquots were 100 ml per 10 g of resin. This procedure was repeated 3 times in order to clean the resin and in order to generate the OH form. A final rinse with water was carried out until the conductivity of the eluting water was 2 μS. The resin was then used to titrate 40 ml of a 50 mg/ml solution of S-[2-[(1-Iminoethyl)amino]ethyl]-2-methyl-L-cysteine di-hydrochloride. The concentration is expressed in terms of zwitterion equivalents. Aliquots were taken throughout the titration, filtered and analyzed by HPLC. Subsamples of the aliquots were saved for a second HPLC analysis 1 week after the titration was performed in order to assess the stability of the samples. Additional aliquots were taken for Cl analysis using an ion selective electrode. The pH was also monitored throughout the titration.

### Example 7

### Preparation of Free Zwitterion

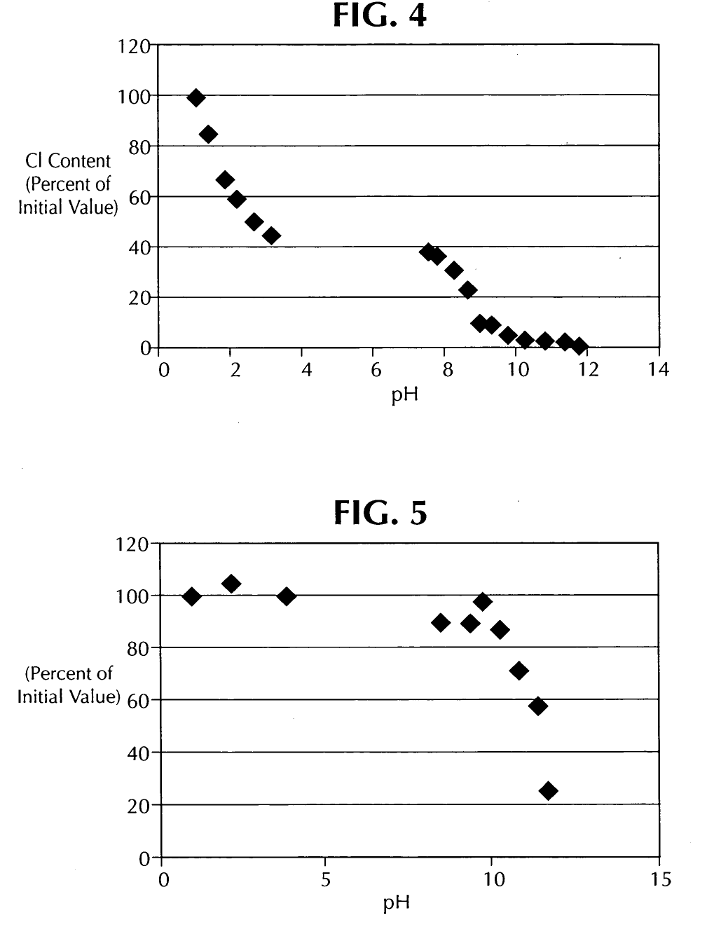

The results found in this example mirrored the results found in Example 3. The chloride specific electrode used here to measure the Cl content produced data that were much less noisy (see FIG. 4). Note that the data indicate that in removing 98% of the Cl a pH of ˜10.85 is reached. More Cl can be removed but this produced significant binding of compound to the resin (see FIG. 5). This loss of compound due to resin binding can be minimized by filtering off the resin toward the end of titration and replacing a small amount of fresh resin. This practice helps drive the equilibrium of chloride removal and minimize the sites available for compound loss by binding.

FIG. 4 Shows titration curves of S-[2-[(1-Iminoethyl)amino]ethyl]-2-methyl-L-cysteine in water with IRA-400 anion exchange resin (Rohm & Haas Amberlite, available from Rohm & Haas, Philadelphia, Pa.). FIG. 5 shows the relevant binding data associated with increasing pH.

HPLC analyses were performed using an ion pairing gradient method. The method has been shown to detect the presence of the degradation products that are expected when S-[2-[(1-Iminoethyl)amino]ethyl]-2-methyl-L-cysteine is made basic. As can be seen in the following table, the data indicate that degradation is not immediate but instead occurs over a period of days.

TABLE 2
Stability of-[2-[(1-Iminoethyl)amino]ethyl]-2-methyl-L-cysteine
free zwitterion.

| | Purity At The | Purity One Week |
|---|---|---|
| | Time Of Titration | After Titration |
| Sample pH | (Peak Area %) | (Peak Area %) |
| .94 | 98.0 | 98.3 |
| 2.13 | 98.6 | 98.4 |
| 3.83 | 98.7 | 98.1 |
| 8.48 | 98.5 | 97.4 |
| 9.37 | 98.5 | 97.2 |
| 9.78 | 98.3 | 97.2 |
| 10.27 | 98.3 | 96.4 |
| 10.83 | 96.6 | 94.7 |
| 11.6 | 98.3 | 92.4 |
| 11.75 | 97.9 | 89.2 |

Samples were analyzed a few hours after preparation of the free zwitterion and again after 1 week.

HPLC Method
Pump A: 20 mM KH₂PO₄, 10 mM Pentane sulfonic acid, adjusted to pH=3 with phosphoric acid
Pump B: Acetonitrile
Gradient: 0% B at 0 min, 26% B at 15 min, 0% B at 15.1 min
Column: YMC ODS-AQ 120 A, 5 μm, 2.6×150 mm
Wavelength=205 nm

### Example 8

### Removal of Excess HCl/Preparation of Monohydrochloro 2-[(1-Iminoethyl)amino]ethyl]-2-methyl-L-cysteine

In these examples, the chloride removal process was run in batch by stirring the resin, but it could easily be run in a plant setting by recirculating the S-[2-[(1-Iminoethyl)amino]ethyl]-2-methyl-L-cysteine dihydrochloride solution over an anion exchange resin column or an anion exchange membrane. If the pH is inadvertently raised beyond the desired range, it may easily be adjusted back by adding an appropriate amount of HCl. It would be well within the ordinary skill in the art to design a large scale anion exchange process for this purpose.

### Example 9

### Crystallization of S-[2-[(1-Iminoethyl)amino]ethyl]-2-methyl-L-cysteine salicylate monohydrate

The first crystallization of S-[2-[(1-Iminoethyl)amino]ethyl]-2-methyl-L-cysteine salicylate monohydrate occurred from a solution containing about 15 mg of zwitterion and 9.5 mg salicylic acid in 250 microliters (μl) of N,N-dimethyl formamide. 2.5 ml of THF was added drop wise with stirring. The solution developed a pearlescent precipitate. Inspection of the precipitate by polarized light microscopy showed birefringent acicular crystals. The precipitate was collected by filtration on a 5.0 micrometer pore size Millipore LS filter. 21 mg of solid was recovered. No attempt was made to thoroughly dry the solid. Elemental analysis [C, H, N] results were close to theory for a one to one monohydrate salt of zwitterion with salicylic acid. Karl Fischer water analysis also indicated a monohydrate stoichiometry.

TABLE 3
Elemental Analysis of S-[2-[(1-Iminoethyl)amino]ethyl]-2-
methyl-L-cysteine salicylate monohydrate crystals

| | Theory 1:1 | |
|---|---|---|
| Element | monohydrate (%) | Measured (%) |
| Carbon | 47.99 | 50.35/50.31 |
| Hydrogen | 6.71 | 7.13/7.12 |
| Nitrogen | 11.19 | 9.76/9.83 |
| Sulfur | 8.54 | — |
| Water by Karl Fischer | 4.79 | 5.0 |

Experiments were designed to obtain enough crystalline material for seeding and more complete characterization. An outline of the experiments conducted to improve upon the crystallization are listed in Table 4. Some of these experiments were seeded with crystalline material from a previous lot. Surprisingly, seeding did not appear to have any effect on the induction or rate of crystallization.

### Example 10

This example was prepared with 505 mg of S-[2-[(1-Iminoethyl)amino]ethyl]-2-methyl-L-cysteine dissolved in 6.0 ml of water and 14 ml of DMF. 100 ml of acetonitrile was added with stirring. After spontaneous crystallization had produced a slurry, an additional 25 ml of acetonitrile was added over 15 minutes. The slurry was stirred for 2 hours longer. The slurry was filtered onto Whatman paper in a Buchner funnel. Solids were dried at 40° C. under house vacuum for about 22 hours. 511 mg of dried solid was recovered.

### Characterization of S-[2-[(1-Iminoethyl)amino]ethyl]-2-methyl-L-cysteine salicylate monohydrate

Examples 9 and 10, starting with 100 milligrams each of S-[2-[(1-Iminoethyl)amino]ethyl]-2-methyl-L-cysteine zwitterion, were crystallized from 400 microliters of water and 1.77 Ml of DMF containing one equivalent of salicylic acid. Example 9 was precipitated with THF, and Example 10 was precipitated with acetonitrile. These solids were collected by filtration and vacuum dried overnight at 40° C. Extremely fine, acicular, birefringent needles with positive elongation were observed in both lots by polarized light microscopy.

A high NA objective and well adjusted Kohler illumination are required to observe these crystals by light microscopy because of their fineness. Collection by filtration was often very slow.

TABLE 5
Elemental Analysis of Salicylate monohydrate
Salt Examples 9 and 10

| | Example 12 | Example 13 | Theory 1:1 |
|---|---|---|---|
| Element | (THF) | (ACN) | monohydrate |
| Carbon | 50.53 | 48.13/48.12 | 47.99 |
| Hydrogen | 7.23 | 6.83/6.88 | 6.71 |
| Nitrogen | 10.77 | 11.53/11.46 | 11.19 |
| Sulfur | 7.96 | 8.61/8.61 | 8.54 |
| Water (KF) | — | 4.75 | 4.79 |

Elemental analysis and Karl Fischer water titration show the salt is a one to one salicylate monohydrate with some propensity to retain solvents. Proton NMR confirms one to one stoichiometry of S-[2-[(1-Iminoethyl)amino]ethyl]-2-methyl-L-cysteine and salicylic acid. Residual DMF was indicated at about 0.10 to 0.17 mole percent in lots analyzed by H-NMR after drying under vacuum at 40° C. overnight. The results of elemental analyses such as those for Example 9, Table 3, can be accounted for by adjustments of included solvent.

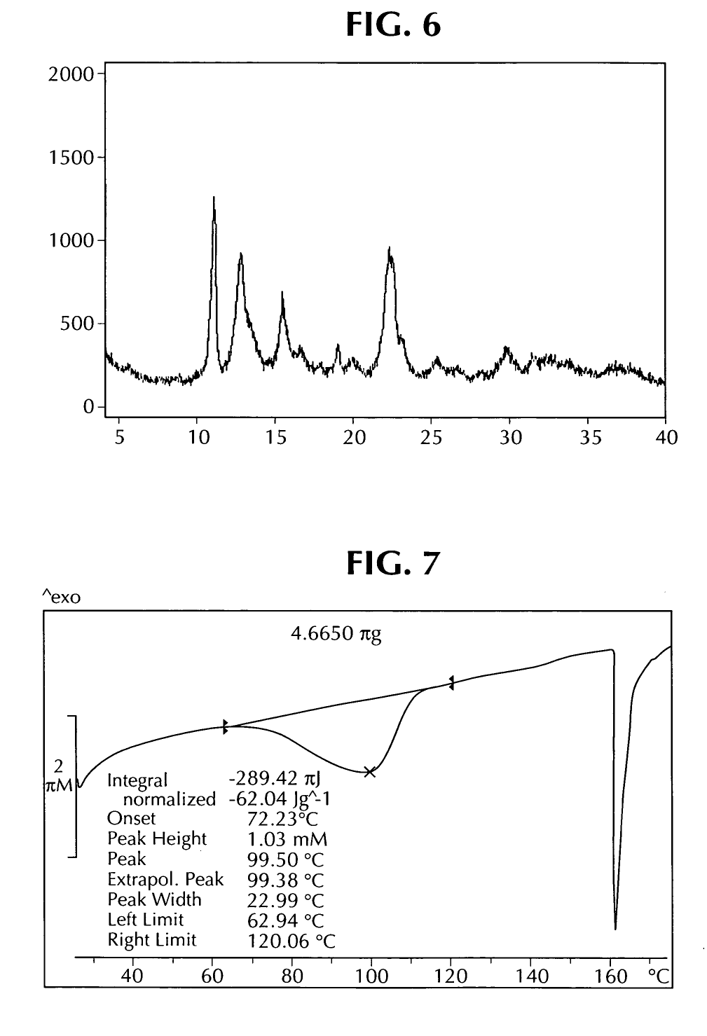

S-[2-[(1-Iminoethyl)amino]ethyl]-2-methyl-L-cysteine salicylate monohydrate is crystalline by x-ray powder diffraction. Powder pattern of Example 10 is shown in FIG. 6.

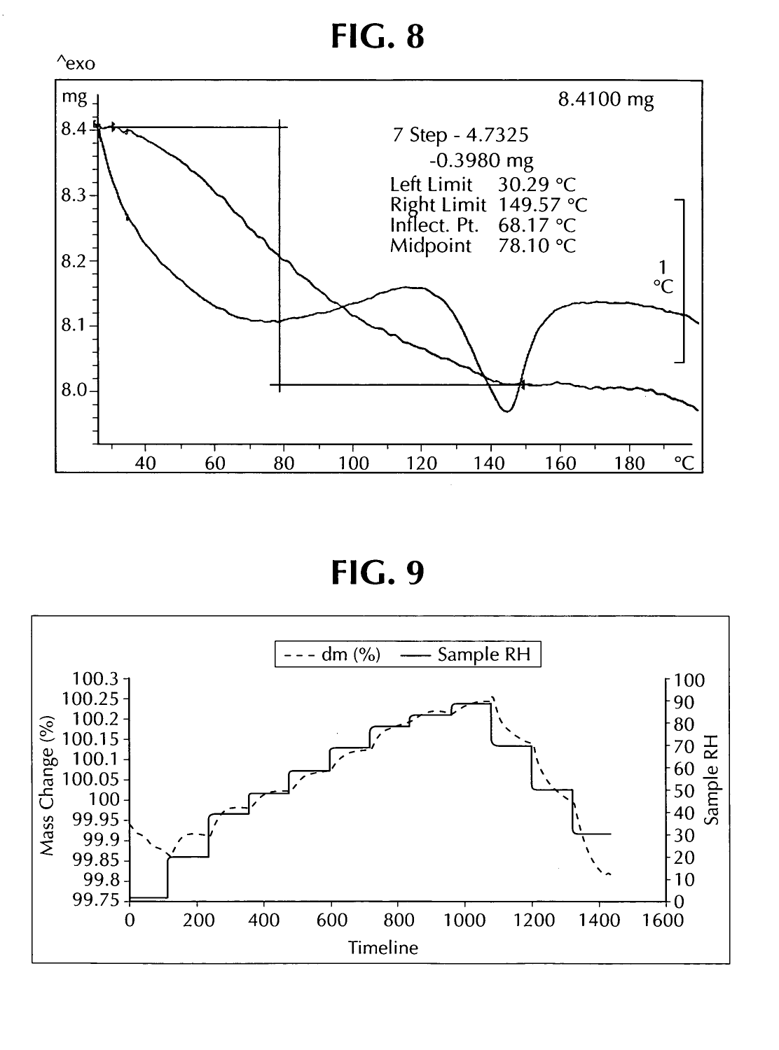

A broad melt appears to occur after loss of the water of crystallization, near 125° C. by hot stage microscopy. The apparent melt by hot stage microscopy, with sample in silicon oil, does not exhibit the typical vapor bubbles from water at 100 to 110° C. DSC, FIG. 7 (Example 9), and SDTA, FIG. 8 (Example 10), curves indicate different melting points. This may indicate that a melt is not being observed, but rather dissolution of the solid in the water of hydration. The transition at 160° C. in the DSC, FIG. 7, is decomposition.

Referring to FIG. 9, the apparent loss of DMF during the moisture balance measurement is reflected in the overall mass loss during the moisture absorption experiment. The salt is non-hygroscopic absorbing less than half of one percent water at 80% RH, (100.25%-99.8% at end).

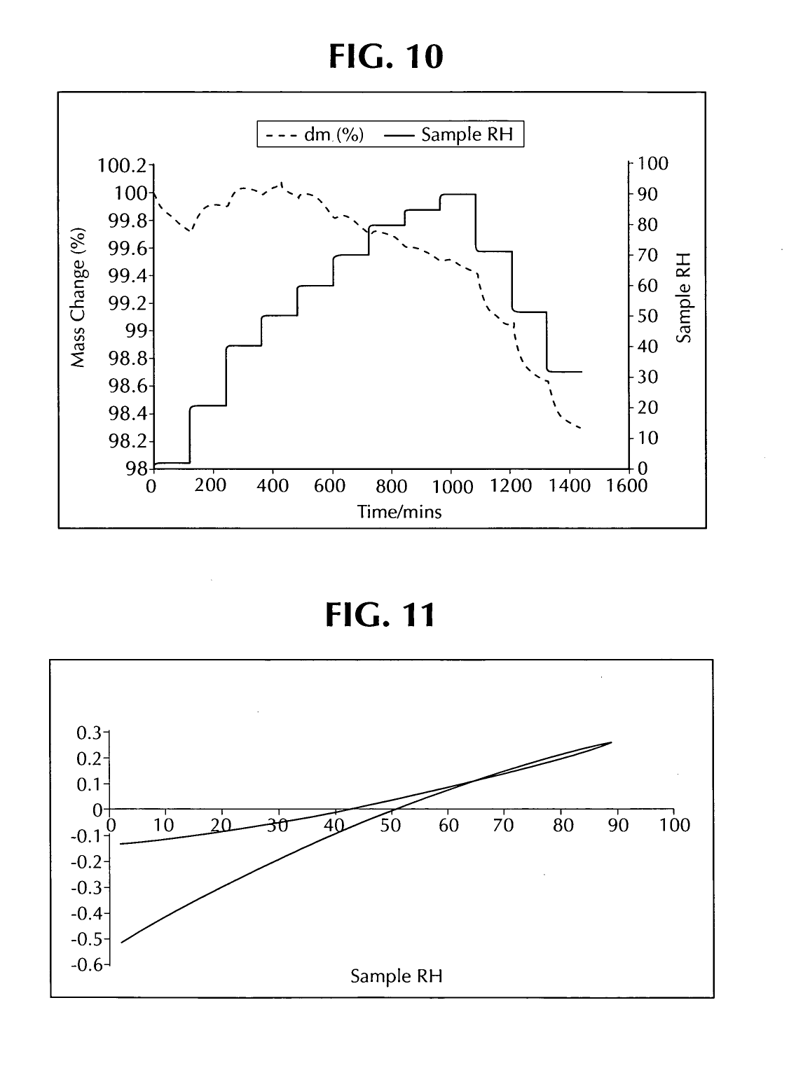

DVS moisture sorption of several lots of the crystalline salt form show it is non-hygroscopic (see FIG. 10). The crystalline salicylate monohydrate salt absorbs less than one percent water by weight at 90% R.H, 25° C. Moisture balance measurements indicate that much of the DMF is removed at 25° C. in flowing moist nitrogen.

The aqueous solubility, in HPLC grade water, is estimated between 135 and 148 mg Ml⁻¹ determined gravimetrically in microcentrifuge tubes.

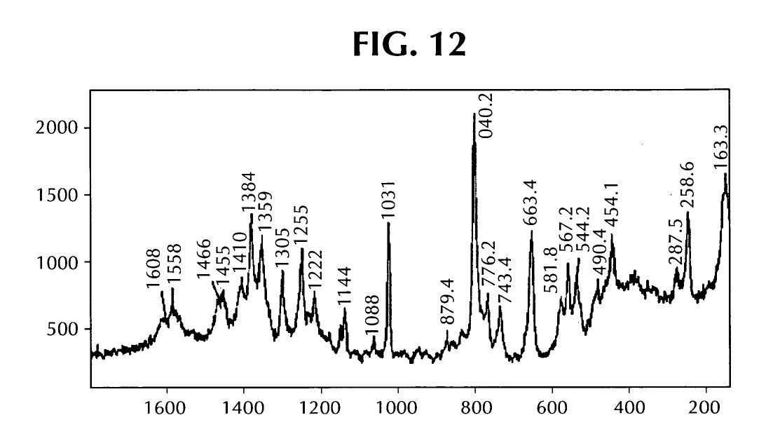

FIG. 12 shows the Raman spectrum of the crystalline S-[2-[(1-Iminoethyl)amino]ethyl]-2-methyl-L-cysteine salicylate monohydrate. Briefly, the Raman spectrum is a vibrational signature of a molecule or complex system. Its origin lies in the inelastic collisions between the molecules and photons, which are the particles of light composing a light beam. The collision between the molecules and the photons leads to an exchange of energy with consequent change in energy and hence wavelength of the photon. Thus, a Raman spectrum is a set of very narrow spectral lines emitted from object molecules when illuminated by an incident light. The width of each spectral line is strongly affected by the spectral width of the incident light and hence tightly monochromatic light sources, such as lasers, are used. The wavelength of each Raman line is expressed as a wave number-shift from the incident light, which is the difference between the inverse wavelength of the Raman line and the incident light. The wave number-shift, not the absolute wavelength, of the Raman lines is specific to particular atomic groups in molecules. Raman spectra measure the vibration states of molecules, which are determined by their molecular structure.

Also embraced within this invention is a class of pharmaceutical compositions comprising crystalline S-[2-[(1-Iminoethyl)amino]ethyl]-2-methyl-L-cysteine salicylate monohydrate in association with one or more non-toxic, pharmaceutically-acceptable carriers and/or diluents and/or adjuvants (collectively referred to herein as “carrier” materials) and, if desired, other active ingredients. The crystalline form of S-[2-[(1-Iminoethyl)amino]ethyl]-2-methyl-L-cysteine salicylate monohydrate of the present invention may be administered by any suitable route, preferably in the form of a pharmaceutical composition adapted to such a route, and in a dose effective for the treatment intended. The active S-[2-[(1-Iminoethyl)amino]ethyl]-2-methyl-L-cysteine salicylate monohydrate and compositions may, for example, be administered orally, intravascularly, intraperitoneally, subcutaneously, intramuscularly or topically.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. The active ingredient may also be administered by injection as a composition wherein, for example, saline, dextrose or water may be used as a suitable carrier.

The amount of therapeutically active compound that is administered and the dosage regimen for treating a disease condition with the compound and/or compositions of this invention depends on a variety of factors, including the age, weight, sex and medical condition of the subject, the severity of the disease, the route and frequency of administration, and the particular compound employed, and thus may vary widely. The pharmaceutical compositions may contain active ingredients in the range of about 0.1 to 2000 mg, preferably in the range of about 0.5 to 500 mg and most preferably between about 1 and 100 mg. A daily dose of about 0.01 to 100 mg/kg body weight, preferably between about 0.5 and about 20 mg/kg body weight and most preferably between about 0.1 to 10 mg/kg body weight, may be appropriate. The daily dose can be administered in one to four doses per day.

Crystalline S-[2-[(1-Iminoethyl)amino]ethyl]-2-methyl-L-cysteine salicylate monohydrate can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0-5 to 20%, advantageously 0.5 to 10% and particularly about 1.5% w/w.

For therapeutic purposes, S-[2-[(1-Iminoethyl)amino]ethyl]-2-methyl-L-cysteine salicylate monohydrate is ordinarily combined with one or more adjuvants appropriate to the indicated route of administration. If administered per os, the compound may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The crystalline S-[2-[(1-Iminoethyl)amino]ethyl]-2-methyl-L-cysteine salicylate monohydrate may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

1. A crystalline form of S-[2-[(1-Iminoethyl)amino]ethyl]-2-methyl-L-cysteine salicylate monohydrate characterized by at least one of: x-ray powder pattern substantially as shown in FIG. 6; Raman spectrum substantially as shown in FIG. 12; and elemental analysis substantially as in Table 5.

2. A pharmaceutical composition comprising the crystalline S-[2-[(1-Iminoethyl)amino]ethyl]-2-methyl-L-cysteine salicylate monohydrate of claim 1 together with a pharmaceutically acceptable carrier.

3. A method of treating a condition wherein pathologically high production forms a part in a subject in need of such treatment comprising administering to the subject an effective amount of the crystalline S-[2-[(1-Iminoethyl)amino]ethyl]-2-methyl-L-cysteine salicylate monohydrate of claim 1.

4. A method of decreasing nitric oxide production in a subject comprising administering to the subject an effective amount of the crystalline S-[2-[(1-Iminoethyl)amino]ethyl]-2-methyl-L-cysteine salicylate monohydrate-of claim 1.

5. A method of making S-[2-[(1-Iminoethyl)amino]ethyl]-2-methyl-L-cysteine salicylate monohydrate comprising:
obtaining S-[2-[(1-Iminoethyl)amino]ethyl]-2-methyl-L-cysteine zwitterion;
adding the S-[2-[(1-Iminoethyl)amino]ethyl]-2-methyl-L-cysteine zwitterion to an appropriate solvent;
adding a salicylic acid to the S-[2-[(1-Iminoethyl)amino]ethyl]-2-methyl-L-cysteine and solvent; and
adding an antisolvent to precipitate S-[2-[(1-Iminoethyl)amino]ethyl]-2-methyl-L-cysteine salicylate monohydrate crystals.

6. The method of claim 5 wherein at least two solvents are used.

7. The method of claim 6 wherein at least one of the two solvents is N,N-dimethylformamide.

8. The method of claim 6 wherein at least one of the two solvents is water.

9. The method of claim 5 wherein the antisolvent is acetonitrile.
